SEQUENCE LISTING (2 sequences)

SEQ ID NO: 1 - DNA, 1557 bp, Escherichia coli
atgatcccggacgtatcacaggcgctggcctggctggaaaaacatcctcaggcgttaaaggggatacagcgtgggctggagcgcgaaactttgcgtgttaatgctgatggcacactggcaacaacaggtcatcctgaagcattaggttccgcactgacgcacaaatggattactaccgattttgcggaagcattgctggaattcattacaccagtggatggtgatattgaacatatgctgacctttatgcgcgatctgcatcgttatacggcgcgcaatatgggcgatgagcggatgtggccgttaagtatgccatgctacatcgcagaaggtcaggacatcgaactggcacagtacggcacttctaacaccggacgctttaaaacgctgtatcgtgaagggctgaaaaatcgctacggcgcgctgatgcaaaccatttccggcgtgcactacaatttctctttgccaatggcattctggcaagcgaagtgcggtgatatctcgggcgctgatgccaaagagaaaatttctgcgggctatttccgcgttatccgcaattactatcgtttcggttgggtcattccttatctgtttggtgcatctccggcgatttgttcttctttcctgcaaggaaaaccaacgtcgctgccgtttgagaaaaccgagtgcggtatgtattacctgccgtatgcgacctctcttcgtttgagcgatctcggctataccaataaatcgcaaagcaatcttggtattaccttcaacgatctttacgagtacgtagcgggccttaaacaggcaatcaaaacgccatcggaagagtacgcgaagattggtattgagaaagacggtaagaggctgcaaatcaacagcaacgtgttgcagattgaaaacgaactgtacgcgccgattcgtccaaaacgcgttacccgcagcggcgagtcgccttctgatgcgctgttacgtggcggcattgaatatattgaagtgcgttcgctggacatcaacccgttctcgccgattggtgtagatgaacagcaggtgcgattcctcgacctgtttatggtctggtgtgcgctggctgatgcaccggaaatgagcagtagcgaacttgcctgtacacgcgttaactggaaccgggtgatcctcgaaggtcgcaaaccgggtctgacgctgggtatcggctgcgaaaccgcacagttcccgttaccgcaggtgggtaaagatctgttccgcgatctgaaacgcgtcgcgcaaacgctggatagtattaacggcggcgaagcgtatcagaaagtgtgtgatgaactggttgcctgcttcgataatcccgatctgactttctctgcccgtatcttaaggtctatgattgatactggtattggcggaacaggcaaagcatttgcagaagcctaccgtaatctgctgcgtgaagagccgctggaaattctgcgcgaagaggattttgtagccgagcgcgaggcgtctgaacgccgtcagcaggaaatggaagccgctgataccgaaccgtttgcggtgtggctggaaaaacacgcctga

SEQ ID NO: 2 - protein, 518 aa, Escherichia coli
MIPDVSQALAWLEKHPQALKGIQRGLERETLRVNADGTLATTGHPEALGSALTHKWITTDFAEALLEFITPVDGDIEHMLTFMRDLHRYTARNMGDERMWPLSMPCYIAEGQDIELAQYGTSNTGRFKTLYREGLKNRYGALMQTISGVHYNFSLPMAFWQAKCGDISGADAKEKISAGYFRVIRNYYRFGWVIPYLFGASPAICSSFLQGKPTSLPFEKTECGMYYLPYATSLRLSDLGYTNKSQSNLGITFNDLYEYVAGLKQAIKTPSEEYAKIGIEKDGKRLQINSNVLQIENELYAPIRPKRVTRSGESPSDALLRGGIEYIEVRSLDINPFSPIGVDEQQVRFLDLFMVWCALADAPEMSSSELACTRVNWNRVILEGRKPGLTLGIGCETAQFPLPQVGKDLFRDLKRVAQTLDSINGGEAYQKVCDELVACFDNPDLTFSARILRSMIDTGIGGTGKAFAEAYRNLLREEPLEILREEDFVAEREASERRQQEMEAADTEPFAVWLEKHA

(19) United States
(12) Patent Application Publication
(10) Pub. No.: US 2025/0270602 A1
(43) Pub. Date: Aug. 28, 2025

(54) STRAIN OF MICROORGANISMS AND PROCESS FOR THE FERMENTATIVE PRODUCTION OF GAMMA-GLUTAMYLTYROSINE AND GAMMA-GLUTAMYLPHENYLALANINE

(71) Applicant: Wacker Chemie AG, Munich (DE)

(72) Inventors: Thomas Schlösser, Burghausen (DE); Marcel Thön, Halle (DE)

(73) Assignee: Wacker Chemie AG, Munich (DE)

(21) Appl. No.: 17/612,799

(22) PCT Filed: May 20, 2019
(86) PCT No.: PCT/EP2019/062957

§ 371 Date: Nov. 19, 2021

(51) Int. Cl. C12P 13/00; C12N 9/00; C12N 15/70

(52) U.S. Cl. CPC C12P 13/005; C12N 9/93; C12N 15/70; C12Y 603/02002; C12Y 603/02003; C12N 2800/101

(57) ABSTRACT

A micro-organism strain and process for fermentative production of the same. The fermentative production of the substances gamma-glutamyltyrosine and gamma-glutamylphenylalanine by a micro-organism strain which is cultured in a fermentation medium. The fermentation medium is removed from the micro-organisms after the fermentation and gamma-glutamyltyrosine and gamma-glutamylphenylalanine are isolated from the fermentation medium. The microorganism strain contains at least one further gene encoding a glutamate-cysteine ligase and is deficient in glutathione synthetase. For the culture, the fermentation medium is admixed with L-tyrosine and L-phenylalanine.

The invention relates to a process for fermentative production of the substances gamma-glutamyltyrosine (γ-Glu-Tyr) and gamma-glutamylphenylalanine (γ-Glu-Phe).

The term Kokumi comes from the Japanese language and does not describe a taste of its own such as sweet, sour, bitter, salty or umami (savory/piquant), but a taste experience with a strong mouthfeel and persistent richness of taste. The Kokumi effect was initially described by Ueda et al. in 1990 (Agri. Biol. Chem. 54, pages 163-169) when aqueous garlic extracts were sensorily assessed in various soups.

In molecular terms, Ohsu et al. (2010, J. Biol. Chem. 285, pages 1016-1022) was able to show that the Kokumi effect is based on the modulation of a calcium-sensing receptor (CaSR) by gamma-glutamyl peptides such as glutathione (γ-Glu-Cys-Gly) or γ-Glu-Val-Gly.

Interestingly, however, it is not only gamma-glutamyl tripeptides that are responsible for the Kokumi taste experience, but also corresponding dipeptides. For instance, it was discovered by Shibata et al. in 2017 (Biosci. Biotechnol. Biochem. 81, pages 2168-2177) that heat-treated soybeans together with glutamate, salt and inosine monophosphate produce a particularly intense Kokumi taste experience. Interestingly, the dipeptides γ-Glu-Tyr and γ-Glu-Phe are responsible for the intense Kokumi effect. It is thus not surprising that γ-Glu-Tyr and γ-Glu-Phe were detected in piquant Comté cheese by Roudot-Algaron et al. as early as in 1994 (J. Dairy Sci. 77, pages 1161-1166).

It is an object of the present invention to provide a fermentation process for production of gamma-glutamyltyrosine (γ-Glu-Tyr) and gamma-glutamylphenylalanine (γ-Glu-Phe).

This object is achieved by a process for fermentative production of gamma-glutamyltyrosine and gamma-glutamylphenylalanine, characterized in that a microorganism strain is cultured in a fermentation medium, the fermentation medium is removed from the cells after the fermentation, and gamma-glutamyltyrosine and gamma-glutamylphenylalanine are isolated from the fermentation medium, wherein the microorganism strain contains at least one further gene encoding a glutamate-cysteine ligase and is deficient in glutathione synthetase.

In principle, suitable starting strains are all microorganism strains that are open to recombinant methods and are culturable by fermentation. Such microorganism strains can be fungi, yeasts or bacteria. The microorganism strain is preferably a bacterial strain, particularly preferably a bacterial strain of the phylogenetic group of Eubacteria. Microorganism strains of the Enterobacteriaceae family are particularly preferred and the bacterial strain is especially preferably a strain of the species Escherichia coli (E. coli).

Preference is given to E. coli strains which are suitable for production of gamma-glutamylcysteine. The construction of such strains is described in patent document US 2014/0342399.

Particular preference is given to the E. coli strain W3110ΔgshB/ptufBₚ-gshA^{ATG}-cysE14-serA2040-orf306 as disclosed in US 2014/0342399 A.

Open reading frame (ORF) refers to that DNA or RNA region that begins with the start codon and leads up to a stop codon and encodes the amino acid sequence of a protein. The ORF is also synonymously referred to as the coding sequence.

ORFs are surrounded by noncoding regions. Gene refers to the DNA segment which contains all the basic information for producing a biologically active RNA. A gene contains the DNA segment from which a single-stranded RNA copy is produced by transcription and the expression signals which are involved in the regulation of this copying process. The expression signals include, for example, at least one promoter, a transcription start site, a translation start site and a ribosome binding site. Furthermore, a terminator and one or more operators are possible as expression signals.

In the case of a functional promoter, the ORF which is under the regulation of said promoter is transcribed into an RNA.

According to the invention, the microorganism strain contains at least one further ORF encoding a glutamate-cysteine ligase. The term “one further ORF” refers to the fact that an ORF encoding a glutamate-cysteine ligase is already present in the wild-type genome and it is not this chromosomal ORF that is meant.

In the context of this invention, the term “one further ORF” means, on the contrary, that:
a) either a wild-type microorganism strain with regard to the glutamate-cysteine ligase ORF is present, i.e., said strain contains the ORF encoded in the wild-type genome that encodes glutamate-cysteine ligase and, in addition, at least one further ORF encoding a glutamate-cysteine ligase.
b) or the microorganism strain according to the invention has no ORF encoding glutamate-cysteine ligase in the wild-type genome, but at least one further ORF encoding a glutamate-cysteine ligase, i.e., the microorganism strain according to the invention only expresses the enzyme of the further ORF(s).

Glutamate-cysteine ligase or gamma-glutamylcysteine synthetase is an enzyme which metabolically produces gamma-glutamylcysteine from the reactants L-cysteine and L-glutamate with consumption of one molecule of ATP. Said enzyme is encoded by a gene which is, for example, designated gshA in E. coli and GSH1 in Saccharomyces cerevisiae (S. cerevisiae). The glutamate-cysteine ligase is preferably the gene product GshA or GshA protein encoded by the coding region of the gshA gene (gshA ORF). The further ORF encoding a glutamate-cysteine ligase is preferably SEQ ID NO. 1 or homologs of this sequence, with the start codon being preferably ATG; particular preference is given to SEQ ID NO. 1.

In the context of the present invention, homologs at the DNA level are those ORFs that have a sequence identity greater than 30%, particularly preferably greater than 70%, in relation to the ORF sequence present in the particular microorganism in its wild-type form that encodes a glutamate-cysteine ligase, especially preferably in relation to SEQ ID NO. 1 in E. coli, and wherein the protein expressed by this DNA sequence has a glutamate-cysteine ligase function (see below for a definition and for detection of functional glutamate-cysteine ligase, i.e., of the presence of a protein having a glutamate-cysteine ligase function).

The determination of sequence identities of DNA sequences is determined by the program “nucleotide blast”, which can be found on the http://blast.ncbi.nlm.nih.gov/ page and which is based on the blastn algorithm. The algorithm parameters used for an alignment of two or more nucleotide sequences were the default parameters. The default general parameters are:
Max target sequences=100;
Short queries=“Automatically adjust parameters for short input sequences”;
Expect Treshold=10;
Word size=28;
Automatically adjust parameters for short input sequences=0.

The corresponding default scoring parameters are:
Match/Mismatch Scores=1,−2;
Gap Costs=Linear.

The GshA protein is preferably a protein having SEQ ID NO. 2 and homologs of this sequence and is particularly preferably a protein having SEQ ID NO. 2.

Homologs at the protein level are proteins having a sequence identity greater than 30%, particularly preferably greater than 70%, in relation to the protein sequence of glutamate-cysteine ligase that is present in the particular microorganism in its wild-type form, especially preferably in relation to SEQ ID NO. 2 in E. coli, and wherein the protein has a glutamate-cysteine ligase function (see below for a definition and for detection of functional glutamate-cysteine ligase, i.e., of the presence of a protein having a glutamate-cysteine ligase function).

For the determination of sequence identities of protein sequences, the program “protein blast”, on the http://blast.ncbi.nlm.nih.gov/ page, is used.

This program is based on the blastp algorithm. The algorithm parameters used for an alignment of two or more protein sequences were the default parameters. The default general parameters are:
Max target sequences=100;
Short queries=“Automatically adjust parameters for short input sequences”;
Expect Treshold=10;
Word size=3;
Automatically adjust parameters for short input sequences=0.

The default scoring parameters are:
Matrix=BLOSUM62;
Gap Costs=Existence: 11 Extension: 1;
Compositional adjustments=Conditional compositional score matrix adjustment.

The function of the glutamate-cysteine ligase protein in a microorganism strain according to the invention can be characterized as follows:
a) Either the presence of functional glutamate-cysteine ligase in the microorganism strain is indirectly detected via the presence of the products γ-Glu-Tyr and γ-Glu-Phe. To detect γ-Glu-Tyr and γ-Glu-Phe, the HPLC-MS method described in Example 4 can be used.
b) Or the presence of functional glutamate-cysteine ligase in the microorganism strain is directly detected via the enzyme function in an in vitro assay. An enzyme assay for detection of glutamate-cysteine ligase activity is described in patent document US 2014/342399 in Example 9.

The aim of the invention is to achieve an increased cellular activity of glutamate-cysteine ligase. According to the invention, this is achieved by an increased expression of glutamate-cysteine ligase and which, in turn, by increasing the copy number of the ORF encoding a glutamate-cysteine ligase.

Furthermore, the expression of the ORF encoding a glutamate-cysteine ligase is preferably increased through the use of suitable promoters.

A promoter which leads to increased protein expression is referred to as a strong promoter. A strong promoter can be cloned into the relevant wild-type gene for expression of the ORF that is present chromosomally in the wild type and encodes a glutamate-cysteine ligase and/or can regulate the expression of the additional or further ORF encoding a glutamate-cysteine ligase. Examples of promoters which lead to strong expression in the microorganism (strong promoters) are known to a person skilled in the art.

In a preferred embodiment, the microorganism strain according to the invention is a wild-type microorganism strain with regard to the glutamate-cysteine ligase ORF, i.e., it contains the ORF encoded in the wild-type genome that encodes glutamate-cysteine ligase, and it additionally contains at least one further plasmid-encoded ORF encoding a glutamate-cysteine ligase under the control of a strong promoter.

Increased protein expression is to be understood to mean that the protein is produced in an increased yield, which means that what is produced using the microorganism strain according to the invention compared with a corresponding wild-type strain is preferably at least 110%, particularly preferably at least 150% and especially preferably at least 200% of the amount of glutamate-cysteine ligase. This means that the yield of glutamate-cysteine ligase protein is preferably at least 1.1 times, particularly preferably at least 1.5 times and especially preferably at least 2 times as high as the yield which can be achieved using corresponding strains which do not contain a further open reading frame encoding a glutamate-cysteine ligase. The corresponding wild-type microorganism strain is characterized in that it is genetically identical to the strain used, but does not contain a further open reading frame encoding a glutamate-cysteine ligase.

The microorganism strain according to the invention can comprise one or more further functional copies of the ORF encoding a glutamate-cysteine ligase. It preferably comprises one to 700 further functional copies of the ORF encoding a glutamate-cysteine ligase, particularly preferably one to 20 further functional copies of the ORF encoding a glutamate-cysteine ligase. Each further or additional ORF can be integrated into the chromosome; however, it can also be present on one or more plasmids.

According to the invention, the microorganism strain is deficient in glutathione synthetase, i.e., no functional glutathione synthetase is produced by the corresponding gene. The glutathione synthetase is preferably the gene product encoded by the gshB gene in E. coli and by the GSH2 gene in S. cerevisiae. The glutathione synthetase is particularly preferably the gene product encoded by the gshB gene in E. coli.

In a preferred embodiment, the microorganism strain contains at least one plasmid which contains at least one, particularly preferably exactly one ORF encoding a glutamate-cysteine ligase under the control of a functional promoter. In this embodiment, the ORF encoding a glutamate-cysteine ligase is expressed on a plasmid. Such a production plasmid allows the production of γ-Glu-Tyr and γ-Glu-Phe in a microorganism strain.

It is preferred that the microorganism strain has the chromosomal gene encoding a glutamate-cysteine ligase. It is particularly preferred that the microorganism strain has the chromosomal gene and at least one further ORF encoding a glutamate-cysteine ligase that is located on a plasmid. This means that the cells produce plasmid-encoded glutamate-cysteine ligase in addition to the glutamate-cysteine ligase produced by the chromosomal gene.

In an alternatively preferred embodiment, a microorganism strain is used in which the chromosomal gene present in the wild type that encodes a glutamate-cysteine ligase has been deleted and one or more additional ORFs encoding a glutamate-cysteine ligase are integrated chromosomally or are introduced via one or more plasmids.

The expression of the ORF encoding a glutamate-cysteine ligase is achieved by homologous or heterologous promoters.

In the context of this invention, a homologous promoter means that what is selected is the promoter which also regulates the expression of glutamate-cysteine ligase in the wild-type gene corresponding to the selected ORF. By contrast, a heterologous promoter means that what is selected is a promoter which does not regulate the expression of glutamate-cysteine ligase in the corresponding wild-type gene.

In the preferred case, the ORF is under the control of the gshA promoter for expression of the GshA protein, i.e., the homologous promoter is used. Heterologous promoters are, for example, the promoter of the gapA gene from E. coli, the promoter of the catB gene from E. coli, the promoter of the tufB gene from E. coli, the promoter of the mppA gene from E. coli, the promoter of the lpp gene from E. coli and the promoter of the proC gene from E. coli. Furthermore, the heterologous lac, tac, trc, lambda, ara or tet promoters are known to a person skilled in the art and can be used for heterologous expression of the ORF encoding a glutamate-cysteine ligase.

The expression of the ORF encoding a glutamate-cysteine ligase in a microorganism strain is preferably achieved by the promoter of the gapA gene, by the promoter of the catB gene, by the promoter of the tufB gene, by the promoter of the mppA gene, by the promoter of the lpp gene from E. coli or by the promoter of the proC gene from E. coli.

Particularly preferably, the expression of the ORF encoding a glutamate-cysteine ligase on a plasmid in E. coli is achieved by the promoter of the tufB gene from E. coli.

The microorganism strain therefore preferably contains at least one plasmid which comprises one of the abovementioned promoters, the promoter(s) being located on the plasmid in such a way that it/they control(s) the expression of the ORF encoding the glutamate-cysteine ligase.

Preferably, the plasmids which can be used are all available DNA molecules open to genetic engineering that are replicated extrachromosomally in the selected microorganism strain, preferably in E. coli, and that comprise a selection marker. For example, plasmids having a high cellular copy number in E. coli (e.g., plasmids of the pUC series, plasmids of the pQE series, plasmids of the pBluescript series), plasmids having a medium copy number in E. coli (e.g., plasmids of the pBR series, plasmids of the pACYC series) or plasmids having a low copy number in E. coli (e.g., pSC101 or pBeloBAC11) can be used.

In the case of S. cerevisiae, extrachomosomally replicating plasmids derived from the 2 μm plasmid can be used. These can be, for example, YEp vectors (yeast episomal plasmids) containing a selectable marker gene and having a copy number of approx. 50 plasmids per cell. Furthermore, YCp vectors having additionally a centromere sequence for the segregation of the chromosomes into the daughter nuclei can also be used. Furthermore, YRp vectors (yeast replicating plasmids) or ARS vectors, as they are also called, containing autonomously replicating sequences (ARS sequences) can also be used. Owing to the presence of eukaryotic origins of replication, these plasmids replicate autonomously, i.e., independently of the chromosomal DNA.

Furthermore, YAC vectors (yeast artificial chromosomes), which behave like independent chromosomes, can also be used in S. cerevisiae.

What can also be used as plasmids are so-called shuttle vectors having multiple different origins of replication, which are each active in a different species. They are therefore multipliable in different microorganisms. For example, shuttle vectors which have a bacterial origin of replication from E. coli and an origin of replication (ARS element) from S. cerevisiae and are thus replicable in both organisms can be used.

Preference is given to using plasmids having a medium copy number in E. coli (e.g., plasmids of the pBR series, plasmids of the pACYC series).

Particular preference is given to using a plasmid of the pACYC series.

A plasmid is, for example, introduced by a common transformation method such as, for example, electroporation or the CaCl₂ method. Plasmid-bearing clones are then selected via antibiotic resistance. Selection markers known to a person skilled in the art are, for example, the chloramphenicol acetyltransferase gene, which mediates resistance to chloramphenicol, the neomycin phosphotransferase gene, which mediates resistance to kanamycin, the tetracycline efflux gene, which mediates resistance to tetracycline, or the β-lactamase gene, which mediates resistance to ampicillin and carbenicillin.

As an alternative to expression by a plasmid, the ORF encoding a glutamate-cysteine ligase can also be integrated into the chromosome of a microorganism strain in addition to a plasmid according to the invention or alone. Systems known to a person skilled in the art with temperate bacteriophages or integrative plasmids or integration via homologous recombination are preferably used as the integration method.

The process is preferably characterized in that the fermentation medium contains at least 50-6000 mg/L L-tyrosine (L-Tyr) and 50-6000 mg/L L-phenylalanine (L-Phe).

The process is particularly preferably characterized in that the fermentation medium contains at least 50-3000 mg/L L-tyrosine (L-Tyr) and 50-3000 mg/L L-phenylalanine (L-Phe).

The microorganism strain according to the invention is cultured by customary methods known to a person skilled in the art in a shake flask or in a bioreactor (fermenter).

Growth of the microorganism strain according to the invention in a fermenter takes place as a continuous culture, as a batch culture or, preferably, as a fed-batch culture.

Sugar, sugar alcohols or organic acids are preferably used as the carbon source. Particular preference is given to using glucose, lactose, sucrose or glycerol, especially preferably glucose, as carbon sources in the process according to the invention.

Preference is given to metering of the carbon source in a form which ensures that the content of carbon source in the fermenter is kept in a range from 0.1 g/L to 50 g/L during fermentation. Particular preference is given to a range from 0.1 g/L to 10 g/L.

Preference is given to using ammonia, ammonium salts or protein hydrolysates as the nitrogen source in the process according to the invention. In the case of use of ammonia as correction agent for pH control, this nitrogen source is regularly additionally metered during fermentation.

Salts of the elements phosphorus, chlorine, sodium, magnesium, nitrogen, potassium, calcium and iron and, in traces (i.e., in μM concentrations), salts of the elements molybdenum, boron, cobalt, manganese, zinc, copper and nickel can be added as further media additives.

Furthermore, organic acids (e.g., acetate, citrate), amino acids (e.g., L-isoleucine, L-methionine, L-phenylalanine, L-tyrosine, L-glutamate) and vitamins (e.g., vitamin B1, vitamin B6, vitamin B12) can be added to the medium.

Yeast extract, tryptone, corn steep liquor, soy flour or malt extract can, for example, be used as complex nutrient sources.

The incubation temperature for growing a strain according to the invention from the Enterobacteriaceae family is preferably 28-37° C.; particular preference is given to an incubation temperature of 30-32° C.

During fermentation, the pH of the fermentation medium is preferably in the pH range from 5.0 to 8.5; particular preference is given to a pH of 7.0.

Preference is given to incubating the microorganism strain according to the invention, preferably a strain from the Enterobacteriaceae family and particularly preferably an E. coli strain, under aerobic, anaerobic or microaerobic culturing conditions over a period of from 6 h to 150 h and within the optimal growth temperature range for the particular strain. Particular preference is given to culturing times between 6 h and 48 h.

Fermentation is preferably carried out under aerobic or microaerobic growth conditions.

According to the invention, a process for fermentative production of γ-Glu-Tyr and γ-Glu-Phe is concerned, the term fermentative production meaning that at least 5 mg of γ-Glu-Tyr and 5 mg of γ-Glu-Phe, preferably at least 10 mg of γ-Glu-Tyr and 10 mg of γ-Glu-Phe and particularly preferably at least 20 mg of γ-Glu-Tyr and 20 mg of γ-Glu-Phe are produced per liter of culture medium after 48 hours, preferably at least 40 hours and particularly preferably at least 24 hours of culturing time.

Preferably, γ-Glu-Tyr and γ-Glu-Phe are purified from the fermentation medium after the removal of the fermentation medium.

The removal of γ-Glu-Tyr and γ-Glu-Phe from the culture can be effected by methods known to a person skilled in the art, such as centrifugation of the medium to remove the cells with subsequent recovery of the γ-Glu-Tyr and γ-Glu-Phe from the fermentation supernatant. The products γ-Glu-Tyr and γ-Glu-Phe can be obtained after subsequent purification, concentration and drying and optionally formulation or complexation.

The γ-Glu-Tyr and γ-Glu-Phe produced in the process according to the invention is detected and quantified by means of, for example, an HPLC-MS method.

The procedure in principle for the production of Kokumi products is preferably as follows:
1. fermenter culture,
   followed by removal of the cells, for example by centrifugation
2. cell-free culture supernatant,
   followed by sterile filtration and/or nanofiltration
3. sterile cell-free culture supernatant
   followed by drying, for example by spray-drying and/or freeze-drying
4. raw Kokumi product
   followed by homogenization, for example by grinding
5. Kokumi product

### EXAMPLES

The invention is described in more detail hereinbelow with reference to example embodiments, without being limited thereby.

In the examples, use was made of the E. coli strain W3110ΔgshB/ptufBₚ-gshA^{ATG}-cysE14-serA2040-orf306 as disclosed in US 2014/0342399 A. The strain name refers to the E. coli W3110 strain (ATCC 27325) with deletion of the gshB gene, the bacterial cells bearing the plasmid shown in US 2014/0342399 in FIG. 10 and comprising:
the gshA gene with the start codon ATG, wherein the gene is under the control of the tufB promoter,
the serA gene (encoding D-3-phosphoglycerate dehydrogenase),
the cysE gene (encoding serine acetyltransferase) and
ORF306 (encoding the cysteine/O-acetylserine exporter EamA).

### Example 1: Shake-Flask Preculture of a γ-Glu-Tyr and γ-Glu-Phe Production Strain for Fermentation

100 mL of sterile LB medium (10 g/L tryptone, 5 g/L yeast extract, 5 g/L NaCl) were prepared with 5 g/L D(+)-glucose monohydrate and then admixed with tetracycline×HCl in a sterile 500 mL Erlenmeyer flask (final concentration of tetracycline×HCl in the shake flask: 0.01 g/L, sterile-filtered).

Using an inoculation loop, sufficient cells of the production strain W3110ΔgshB/ptufBₚ-gshA^{ATG}-cysE14-serA2040-orf306 were removed from the agar plate and transferred into the supplemented LB medium until at most a weak turbidity of the supplemented LB medium was discernible. This preculture was cultured for 5 h at 32° C. and 130 rpm on a CERTOMAT® IS incubation shaker (Sartorius Stedium Biotech, Göttingen, Germany).

### Example 2: Pre-Fermenter for the Production of γ-Glu-Tyr and γ-Glu-Phe

The pre-fermentation was carried out in a 2 L DASGIP fermenter from Eppendorf (Hamburg, Germany).

100 mL of LB preculture from Example 1 were used for inoculation of the 900 mL of production medium, consisting of 30 g/L D(+)-glucose monohydrate, 0.03 g/L pyridoxine×HCl,0.005 g/L thiamine×HCl, 0.01 g/L tetracycline×HCl, 5 g/L (NH₄)₂SO₄, 0.5 g/L NaCl, 0.9 g/L L-isoleucine, 0.6 g/L D/L-methionine, 5 g/L corn steep solids (e.g., from Sigma-Alldrich), 0.225 g/L CaCl₂×2 H₂O, 0.6 g/L MgSO₄×7 H₂O, 0.15 g/L Na₂MO₄×2 H₂O, 0.3 g/L H₃BO₃, 0.2 g/L CoCl₂×6 H₂O, 0.25 g/L CuSO₄×5 H₂O, 1.6 g/L MnCl₂×4 H₂O, 1.35 g/L ZnSO₄×7 H₂O, 0.075 g/L FeSO₄×7 H₂O, 1 g/L Na₃ citrate×2 H₂O and 1.71 g/L KH₂PO₄. The media constituents were initially charged in a 2 L fermenter under sterile conditions and adjusted to pH=7.0 using sterile correction agents (25% ammonia & 6.8 N H₃PO₄). During culturing for 48 h, the temperature was kept constant at 32° C. and the pH was kept constant at 7.0 by the correction agent (25% ammonia). Struktol J673 (Schill+Seilacher, Hamburg) in a 1:10 dilution in sterile water was used as antifoam agent. The culture was aerated with sterile compressed air at 100 L/h and stirred at a stirrer speed of 450 rpm. After the oxygen saturation had fallen to a value of 50%, the speed was increased via the control unit of the fermenter up to a value of 1450 rpm in order to obtain 30% oxygen saturation. The oxygen saturation was determined using a pO₂ probe which was calibrated to 100% saturation at 450 rpm. Once the glucose content in the fermenter had dropped to approx. 1-2 g/L, a 56% (w/v) D(+)-glucose monohydrate solution was metered in. The feeding of glucose was effected at a flow rate of 10-15 mL/h, with the glucose concentration in the fermenter being kept constant between 0.1 g/L and 10 g/L. The determination of glucose was carried out using the YSI 7100 MBS analyzer (YSI, Yellow Springs, Ohio, USA).

### Example 3: Fermentative Production of γ-Glu-Tyr and γ-Glu-Phe

The production of γ-Glu-Tyr and γ-Glu-Phe in a 2 L DASGIP fermenter from Eppendorf (Hamburg, Germany) was carried out in two independent batches.

The following description of the experimental procedure applies to each of the two batches 1 and 2:

100 mL of the preculture from Example 2 were used for inoculation of the 900 mL production medium, consisting of 5 g/L D(+)-glucose monohydrate, 0.03 g/L pyridoxine×HCl, 0.005 g/L thiamine×HCl, 0.01 g/L tetracycline×HCl, 5 g/L (NH₄)₂SO₄, 0.5 g/L NaCl, 0.9 g/L L- isoleucine, 0.6 g/L D/L-methionine, 30 g/L corn steep solids, 0.225 g/L CaCl₂×2 H₂O, 0.6 g/L MgSO₄×7 H₂O, 0.15 g/L Na₂MO₄×2 H₂O, 0.3 g/L H₃BO₃, 0.2 g/L CoCl₂×6 H₂O, 0.25 g/L CuSO₄×5 H₂O, 1.6 g/L MnCl₂×4 H₂O, 1.35 g/L ZnSO₄×7 H₂O, 0.075 g/L FeSO₄×7 H₂O, 1 g/L Na₃ citrate×2 H₂O and 1.71 g/L KH₂PO₄. The media constituents were initially charged in a 2 L fermenter under sterile conditions and adjusted to pH=6.9 using sterile correction agents (25% ammonia & 6.8 N H₃PO₄). During culturing for 48 h, the temperature was kept constant at 32° C. and the pH was kept constant at 7.0 by the correction agent (25% ammonia). Struktol J673 (Schill+Seilacher, Hamburg) in a 1:10 dilution in sterile water was used as antifoam agent. The culture was aerated with sterile compressed air at 100 L/h and stirred at a stirrer speed of 450 rpm. After the oxygen saturation had fallen to a value of 50%, the speed was increased via the control unit of the fermenter up to a value of 1450 rpm in order to obtain 30% oxygen saturation. The oxygen saturation was determined using a pO₂ probe which was calibrated to 100% saturation at 450 rpm. Once the glucose content in the fermenter had dropped to approx. 1-2 g/L, a 56% (w/v) glucose solution was metered in. The feeding of glucose was effected at a flow rate of 10-15 mL/h, with the glucose concentration in the fermenter being kept constant between 0.1 g/L and 10 g/L. The determination of glucose was carried out using the YSI 7100 MBS analyzer (YSI, Yellow Springs, Ohio, USA). For efficient production of the products γ-Glu-Tyr and γ-Glu-Phe, L-Tyr and L-Phe were metered in via the glucose feed. The supplementary feeding was effected 2 h after the start of culturing at a rate of 10-15 mL/h, with the feed solution having a concentration of 6 g/L L-Tyr and 6 g/L L-Phe.

In addition to the feeding of glucose, potassium glutamate and ammonium thiosulfate [(NH4)₂S₂O₃] were metered in via a combined feed. The supplementary feeding was effected 2 h after the start of culturing at a rate of 4-4.5 mL/h, with the concentration of ammonium thiosulfate and the concentration of potassium glutamate in the feed solution being 73 g/L and 21 g/L, respectively.

The results of the γ-Glu-Tyr and γ-Glu-Phe fermentations using the production strain W3110ΔgshB/ptufBₚ-gshA^{ATG}-cysE14-serA2040-orf306 are combined in Tables 1 (1st batch) and 2 (2nd batch).

TABLE 1
Content of γ-Glu-Tyr and γ-Glu-Phe in the culture
supernatant of the 1st batch after fermentation of the strain
W3110ΔgshB/ptufBₚ-gshA^{ATG}-cysE14-serA2040-orf306

Culturing time

| Product | 24 h | 48 h |
|---|---|---|
| γ-Glu-Tyr [mg/L] | 29 | 32 |
| γ-Glu-Phe [mg/L] | 190 | 610 |

TABLE 2
Content of γ-Glu-Tyr and γ-Glu-Phe in the culture
supernatant of the 2nd batch after fermentation of the strain
W3110ΔgshB/ptufBₚ-gshA^{ATG}-cysE14-serA2040-orf306

Culturing time

| Product | 24 h | 48 h |
|---|---|---|
| γ-Glu-Tyr [mg/L] | 23 | 240 |
| γ-Glu-Phe [mg/L] | 22 | 118 |

### Detection of γ-Glu-Tyr and γ-Glu-Phe

The determination of γ-Glu-Tyr and γ-Glu-Phe was carried out by means of an HPLC-MS method. The analysis was carried out on an Ultimate 3000 HPLC system from Thermo Fisher Scientific (Dreieich, Germany) coupled to an Amazon SL mass spectrometer from Bruker Daltonik (Bremen, Germany). The separation column used was a Nucleoshell® Bluebird RP 18 column, 2.7 μm (100 mm×3.0 mm) from Macherey Nagel (Düren, Germany). The samples were subjected to gradient elution (mobile phase A: water containing 0.1% (v/v) acetic acid, mobile phase B: methanol) at 30° C. and a flow rate of 0.7 mL/min. The detection by mass spectrometry was effected in ESI positive mode on the basis of the [M+H]⁺ ions (m/z for γ-Glu-Tyr: 311, m/z for γ-Glu-Phe: 295).

### Example 5: Production of a Kokumi Product

To produce a Kokumi product, 1.5 L of a fermentation as described in Example 3 were worked up. After centrifugation to remove the cells, the supernatant was sterile-filtered (Thermo Scientific™ Nalgene™ Rapid-Flow™ 90 mm 0.2μ Filter Unit, Dreieich, Germany) and then dried in a drying cabinet (Binder ED115 E2, Tuttlingen, Germany). For homogenization, the dried raw product was homogenized in a mill (IKA® All basic, Staufen, Germany).

1-7. (canceled)

8. A process for fermentative production of gamma-glutamyltyrosine and gamma-glutamylphenylalanine: comprising:
providing a bacterial strain of the Enterobacteriaceae family that is cultured in a fermentation medium, wherein the fermentation medium is removed from the cells after the fermentation, and gamma-glutamyltyrosine and gamma-glutamylphenylalanine are isolated from the fermentation medium,
wherein the bacterial strain
a) either contains an open reading frame encoded in the wild-type genome that encodes glutamate-cysteine ligase and, in addition, at least one further open reading frame (ORF) encoding a glutamate-cysteine ligase, or
b) contains no ORF encoded in the wild-type genome that encodes a glutamate-cysteine ligase, but at least one further ORF encoding a glutamate-cysteine ligase;
wherein the bacterial strain is deficient in glutathione synthetase, i.e., no functional glutathione synthetase is produced by the corresponding gene; and
wherein the fermentation medium contains at least 50 mg/L L-tyrosine and 50 mg/L L-phenylalanine.

9. The process of claim 8, wherein the bacterial strain is a strain of the species Escherichia coli.

10. The process of claim 8, wherein the at least one further open reading frame encoding a glutamate-cysteine ligase is SEQ ID NO. 1 or homologs of this sequence; and
wherein the homologs are those ORFs that have a sequence identity greater than 70% in relation to SEQ ID NO. 1 and the protein expressed by this DNA sequence has a glutamate-cysteine ligase function.

11. The process of claim 8, wherein the bacterial strain contains at least one plasmid which contains at least one ORF encoding a glutamate-cysteine ligase under the control of a functional promoter.

12. The process of claim 8, wherein the gamma-glutamyltyrosine and gamma-glutamylphenylalanine are purified from the fermentation medium after the removal of the fermentation medium.
